(19) United States
(12) Patent Application Publication
(10) Pub. No.: US 2020/0405797 A1
(43) Pub. Date: Dec. 31, 2020

(54) TRANSMUCOSAL DELIVERY DEVICE AND METHOD OF MANUFACTURING SAME

(71) Applicant: Nanostrips, Inc., Irvine, CA (US)

(72) Inventor: John David Sanderson, Irvine, CA (US)

(73) Assignee: Nanostrips, Inc., Irvine, CA (US)

(21) Appl. No.: 16/955,674

(22) PCT Filed: Dec. 18, 2018
(86) PCT No.: PCT/US2018/066253

§ 371 Date: Jun. 18, 2020

Related U.S. Application Data
(60) Provisional application No. 62/607,236, filed on Dec. 18, 2017.

(51) Int. Cl. A61K 36/185; A61K 31/352; A61K 47/28; A61K 9/51; A61K 47/26; A61K 9/70

(52) U.S. Cl. CPC A61K 36/185; A61K 31/352; A61K 9/7007; A61K 9/5123; A61K 47/26; A61K 47/28

(57) ABSTRACT

A transmucosal delivery device comprising a mucosal permeation enhancing agent and a nanocarrier carrying a biologically active substance are embedded within a colloidal polymer thin film. Such a transmucosal delivery device is intended to deliver the biological active substance to a target location in a two-stage process. First, the colloidal polymer thin film is dissolved when adhered to a mucosal membrane. Once absorbed through the mucosal tissue and into system circulation, the nanocarrier is subject to a number of physiologic effects that will lead to the nanocarrier reaching a target location, such as a target tissue, wherein the biologically active substance dissolves and the biologically active substance payload is released.

### FIELD

This disclosure relates generally to a transmucosal delivery device for delivering a biologically active substance and a method of manufacturing same.

### BACKGROUND

Within the oral mucosal cavity, the buccal region offers an attractive route of administration for systemic delivery of biologically active substances, including both synthetic and naturally occurring molecules. The buccal mucosa is a barrier, providing protection to underlying tissue. The buccal mucosa has a rich blood supply and it is relatively permeable, and in particular, is more permeable than other barriers such as skin. The buccal mucosa offers several advantages for controlled substance delivery for extended periods of time compared to other known methods such as gastrointestinal ingestion. The mucosa is well supplied with both vascular and lymphatic drainage. Substances gaining entry into the venous plexus of oral mucous membranes are transported back to the heart for system distribution. This contrasts with pre-system elimination and first-pass metabolism in the liver when substances gain entry via a swallowing (gastrointestinal) route of ingestion, including ease of administration, improved compliance, and improved bioavailability.

Known transmucosal delivery devices include thin films generally comprising a thin, flexible polymer, with or without a plasticizer, and with an active substance dispersed within a polymeric colloid. These thin films comprise polymeric matrices and are typically manufactured by solvent casting and are designed to have muco-adhesive properties. These thin films are also known to be formulated with permeability enhancers to improve bioavailability. Known oral thin film delivery devices have shown capabilities that improve the onset of action, reduce the dose frequency and enhance the efficacy of the bioactive substance.

However, known transmucosal thin film delivery devices have room for improvement, such as in the areas of bioactive loading capacity, dissolution rate, residence time at site of administration, formulation stability, toxicity, biocompatibility, and biodegradability. It is therefore an objective to provide an improvement to known transmucosal thin film delivery devices.

### SUMMARY

According to one aspect of the invention, there is provided a transmucosal delivery device comprising: a colloidal polymer thin film; a nanocarrier embedded in the collodial polymer thin firm and carrying a biologically active substance; and a permeation enhancing agent embedded in or coating the polymer thin film. The nanorcarrier can be a nano-bilosome. The biologically active material can comprise a cannabis extract, or a synthetic agonist of cannabinoid receptors, which can contain a tetrahydrocannabinol in concentrations of 0% to 95% W/V, a cannabinoid in concentrations between 0 and 95% W/V, and a terpene in concentrations between 0% and 25% W/V. The mucosal permeation enhancing agent can comprise one or more of bile acids and salts selected from a group consisting of: taurocholic, glycocholic, taurochenodeoxycholic, glycochenodeoxycholic, chenodeoxycholic acid, deoxycholic acid, lithocholic, cholic acid, chenodeoxycholic acid, deoxycholic acid, conjugated salts of their 7-alpha-dehydroxylated derivatives, deoxycholic acid and lithocholic acid, and derivatives of cholic, chenodeoxycholic and deoxycholic acids.

The biologically active material can comprise a plant, animal, or microorganism extract selected from a group consisting of: iris extract, ashitaba extract, thujopsis dolobrata extract, asparagus extract, avocado extract, sweet hydrangea leaf extract, almond extract, altea extract, arnica extract, aloe extract, apricot extract, apricot kernel extract, ginkgo extract, inchikow extract, fennel extract, turmeric extract, oolong tea extract, uva-ursi extract, rose fruit extract, echinacea leaf extract, isodon japonicus extract, scutellaria root extract, phellodendron bark extract, coptis japonica extract, barley extract, panax ginseng extract, hypericum extract, lamium album extract, ononis extract, Netherland mustard extract, orange extract, a dried seawater product, seaweed extract, persimmon leaf extract, pyracantha fortuneana fruit extract, hydrolyzed elastin, a hydrolyzed wheat powder, hydrolyzed silk, pueraria root extract, chamomilla extract, oil-soluble chamomilla extract, carrot extract, capillary artemisia extract, wild oat extract, hibiscus sabdariffa extract, licorice extract, oil-soluble licorice extract, kiwi extract, kiou apple extract, Jew's ear mushroom extract, cinchona bark extract, cucumber extract, paulownia tomentosa leaf extract, guanosine, guava extract, sophora root extract gardenia extract oil, kuma bamboo grass extract, sophora flavescens extract, walnut extract oil, chestnut extract, grapefruit extract, clematis extract, black rice extract, brown sugar extract, black vinegar extract, chlorella extract, mulberry extract, gentian extract, geranium thunbergii extract, black tea extract, yeast extract, magnolia bark extract, coffee extract burdock extract, rice extract, fermented rice extract, fermented rice bran extract, rice germ, comfrey extract, collagen, cowb extract, saisin extract, bupleurum root extract, umbilical cord extract liquid, saffron extract, salvia extract, saponaria officinalis extract, sasa extract, hawthorn extract sansha extract, Japanese pepper extract, shiitake mushroom extract, rehmannia root extract, lithospermum root extract, perilla extract, Japanese linden extract, meadowsweet extract, jatoba extract, peony extract, ginger extract, calamus root extract, white birch extract, white Jew's ear mushroom extract, horsetail extract, stevia extract, a stevia-fermented product, Chinese tamarisk extract, English ivy extract, whitethorn extract, Sambucus nigra extract, yarrow extract, peppermint extract, sage extract, mallow extract, cnidium rhizome extract, Japanese green gentian extract, mulberry bark extract, rhubarb extract, soybean extract, jujube extract, thyme extract, dandelion extract, lichens extract, tea extract, clove extract, imperata extract, citrus unshiu peel extract, tea tree extract, Chinese blackberry extract, capsicum extract, Japanese angelica root extract, calendula officinalis extract, peach kernel extract, spruce extract, houttuynia extract, tomato extract, natto extract, carrot extract, garlic extract, eglantine extract, hibiscus extract, ophiopogon extract, lotus extract, parsley extract, birch extract, honey, hamamelis extract, parietaria extract, isodon japonicus extract, bisabolol, cypress extract, lactobacillus bifidus extract, loquat extract, coltsfoot extract, petasites japonicus extract, hoelen extract, butcher bloom extract, grape extract, grape seed extract, propolis, loofah extract, safflower extract, peppermint extract, linden extract, tree peony extract, hop extract, rosa rugosa extract, pine extract, horse chestnut extract, skunk cabbage extract, soapberry extract, melissa extract, mozuku extract, peach extract, cornflower extract, eucalyptus extract, saxifrage extract, yuzu extract, lily extract, coix seed extract, wormwood extract, lavender extract, green tea extract, egg shell membrane extract, apple extract, rooibos tea extract, litchi extract, lettuce extract, lemon extract, weeping forsythia extract, astragalus sinicus extract, rose extract, rosemary extract, roman chamomile extract, royal jelly extract, and burnet extract.

Alternatively, the biologically active material can comprise a plant-derived polyphenol selected from a group consisting of: acacetin, apiin, apigenin, apigetrin, artoindonesianin P, baicalein, baicalin, chrysin, cynaroside, diosmetin, diosmin, eupatilin, flavoxate, 6-hydroxyflavone, genkwanin, hidrosmin, luteolin, nepetin, nepitrin (nepetin 7-glucoside), nobiletin, orientin (isoorientin), oroxindin, oroxylin A, rhoifolin, scutellarein, scutellarin, tangeritin, techtochrysin, tetuin, tricin, veronicastroside, vitexin (isovitexin), and wogonin, a flavonol including 3-hydroxyflavone, azaleatin, fisetin, galangin, gossypetin, kaempferide, kaempferol, isorhamnetin, morin, myricetin, natsudaidain, pachypodol, quercetin, rhamnazin, rhamnetin, and sophorin, a flavanone including butin, eriodictyol, hesperetin, hesperidin, homoeriodictyol, isosakuranetin, naringenin, naringin, pinocembrin, poncirin, sakuranetin, sakuranin, and sterubin, dihydroquercetin), and aromadedrin, alpinumisoflavone, anagyroidisoflavone A and B, calycosin, daidzein, daidzin, derrubone, di-O-methylalpinumisoflavone, formononetin, genistein, genistin, glycitein, ipriflavone, irigenin, iridin, irilone, 4′-methyl-alpinumisoflavone, 5-O-methylgenistein, luteone, ononin, orobol, pratensein, prunetin, pseudobaptigenin, psi-tectorigenin, puerarin, retusin, tectoridin, tectorigenin, and wighteone, 4-arylcoumarins (neoflavones), 4-arylchromanes, dalbergiones and dalbergiquinols, calophyllolide, coutareagenin, dalbergichromene, dalbergin, and nivetin, afzelechin, arthromerin A, arthromerin B, catechin, epicatechin, epigallocatechin, epicatechin gallate, epigallocatechin gallate, epigallocatechin gallate, epiafzelechin, fisetinidol, gallocatechin, gallocatechin gallate, guibourtinidol, meciadanol (3-O-methylcatechin), mesquitol, propyl gallate, robinetinidol, thearubigin, apiforol and luteoforol, leucocyanidin, leucodelphinidin, leucomalvidin, leucopelargonidin, leucopeonidin, leucorobinetinidin, melacacidin, antirrhinin, apigeninidin, aurantinidin, capensinidin, chrysanthenin, columnidin, commelinin, cyanidin, 6-hydroxycyanidin, cyanidin-3-(di-p coumarylglucoside)-5-glucoside, cyanosalvianin, delphinidin, diosmetinidin, europinidin, fisetinidin, gesneridin, guibourtinidin, hirsutidin, luteolinidin, malvidin, 5-desoxy-malvidin, malvin, myrtillin, oenin, peonidin, 5-desoxy-peonidin, pelargonidin, petunidin, primulin, protocyanin, protodelphin, pulchellidin, pulchellidin 3-glucoside, pulchellidin 3-rhamnoside, robinetinidin, rosinidin, tricetinidin, tulipanin, violdelphin, protocatechuic acid, gallic acid p-caffeic acid, chlorogenic acid, coumaric acid, cyanidin, pelargonidin, peonidin, peonidin malvidin, quercetin, kaempferol, myricetin, apigenin, luteolin, hesperetin, naringenin, eriodictyol, genistein, glycitein, apigenin, luteolin, resveratrol, curcumin, and curcuminoids.

The mucosal permeation enhancing agent can be selected from a group consisting of: 23-lauryl ether, aprotinin, azone, benzalkonium chloride, cetylpyridinium chloride, cetyltrimethylammonium bromide, cyclodextrin, dextran sulfate, lauric acid, lauric acid/propylene glycol, lysophosphatidylcholine, menthol, methoxysalicylate, methyloleate, oleic acid, piperine, bile acids and their salts, phosphatidylcholine, polyoxyetheylene, polysorbate 80, sodium EDTA, sodium glycocholate, sodium glycodeoxycholoate, sodium lauryl sufate, sodium salicylate, sodium taurocholate, sodium taurodeoxycholoate, sulfoxoides, and alkyl glycosides.

The nanocarrier can comprise lipid-nanoparticles selected from a group consisting of solid lipid nanoparticles and nanostructured lipid carriers. The solid lipid nanoparticles can comprise a lipid component selected from a group consisting of: Tristearin, stearic acid, cetyl palmitate, Precirol®, ATO 5, Compritol®, 888 ATO, Dynasan® 116, Dynasan® 118, Softisan® 154, Cutina® CP, Imwitor® 900 P, Geleol®, Gelot® 64, Emulcire® 61, solid triglycerides, trilaurin, tricaprylin, tripalmitin, tristearin, glyceryl trilaurate, glyceryl trimyristate, glyceryl trimyristin, glyceryl tripalmitate, glyceryl tristearate, glyceryl behenate, glyceryl tribehenin, solid diglycerides, dipalmitin, distearin, solid monoglycerides, glyceryl monostearate, glyceryl palmitostearate, glyceryl stearate citrate, long-chain aliphatic alcohols, cetyl alcohol, stearic alcohol, medium and long-chain fatty acids (C10-C22), stearic acid, palmitic acid, behenic acid, capric acid, fatty alcohol esters with long and medium chain fatty acids with polyols (C10-C22), fatty alcohol esters of long-chain fatty acids, cetyl palmitate, cetearyl olivate, hydroxyoctacosanyl hydroxystearate, sterols, cholesterol, cholesterol esters, cholesteryl hemisuccinate, cholesteryl butyrate, cholesterol palmitate, fatty amines, stearyl amine, waxes, beeswax, shea butter, cocoa butter, carnauba wax, ozokerite wax, paraffin wax, ceramides, hydrogenated vegetable oils, hydrogenated castor oil, quaternary ammonium derivatives, behenyl trimethyl ammonium chloride, and/or mixtures thereof. The solid lipid nanoparticles can also comprise a surfactant component comprising a hydrophilic emulsifier selected from a group consisting of: Iuronic® F68 (poloxamer 188) , Pluronic® F127 (poloxamer 407), Tween 20™, Tween 40™, Tween 80™, polyvinyl alcohol, Solutol® HS15, trehalose, sodium deoxycholate, sodium glycocholate, sodium oleate, and polyglycerol methyl glucose distearate.

The solid lipid nanoparticles can comprise a surfactant component comprising a liophilic emulsifier selected from a group consisting of: Myverol® 18-04K, Span 20™, Span 40™, and Span 60™. Alternatively, the solid lipid nanoparticles can comprise a surfactant component comprising an amphiphilic emulsifier selected from a group consisting of: egg lecithin, soya lecithin, phosphatidylcholines, phosphatidylethanolamines, and Gelucire® 50/13.

The nanostructured lipid carriers can comprise a liquid phase lipid selected from a group consisting of: medium chain triglycerides, paraffin oil, 2-octyl dodecenaol, oleic acid, squalene, isopropyl myristate, oils formed by extraction of the oil fraction of plants and fish oils, algae oils, marine oils, oils derived from petroleum, short-chain fatty alcohols, medium-chain aliphatic branched fatty alcohols, fatty acid esters with short-chain alcohols, isopropyl myristate, isopropyl palmitate, isopropyl stearate, dibutyl adipate, medium chain triglycerides, capric and caprylic acid triglycerides, Ci2-C16 octanoates, fatty alcohol ethers, Vitamin E, Miglyol® 912, Transcutol® HP, Labrafil Lipofile® WL 1340, Labrafac® PG, Lauroglycol® FCC, and Capryol® 90.

The transmucosal delivery device can further comprise a mucoadhesive enhancing agent embedded in the polymer thin film, and selected from a group consisting of: Carbopol 971, Carbopol 974, Carbopol 980, Carbopol 940, Carbopol 941, Carbopol 1382, carboxymethlycellulose and salts thereof, hydroxyproplylmethylcelluslose and salts thereof, xanthan gums, polycarbophil, and mixtures thereof.

According to another aspect, there is provided a method for manufacturing a transmucosal delivery device comprising: selecting a biologically active material; admixing a lipid component, a surfactant component, and the biologically active material to form a lipid nanocarrier mixture; subjecting the lipid nanocarrier mixture to shear forces sufficient to create a lipid encapsulated bioactive nano-emulsion; combining the bioactive nano-emulsion with a polymeric precursor base solution to form a hydrated thin film polymer composition; and dehydrating the thin film polymer composition to form a strip. An excipient component can be admixed with the lipid component, surfactant component, and biologically active material. The shear forces can be provided by a process selected from a group consisting of: high pressure homogenization, solvent emulsification, evaporation or diffusion, supercritical fluid extraction of emulsions, and ultrasonication. The polymeric precursor base solution can be composed of a polymerized water soluble polysaccharide, or combination of polysaccharides, a plasticizer, stabilizers and emulsifiers, and water.

The method can further comprise adding a permeation enhancing agent to the hydrated thin film polymer composition. Alternatively, the method can further comprise applying a layer comprising a permeation enhancing agent to the surface of the thin film polymer composition during the dehydrating.

### DRAWINGS

FIG. 1 is a flow chart of steps for manufacturing a transmucosal delivery device according to embodiments of the invention.

FIGS. 2(a) and (b) are respective schematic views of (a) a transmucosal delivery device comprising a colloid polymer thin film with nanodispersed nanocarriers and embedded permeation enhancing agents according to one embodiment of the invention, and (b) a transmucosal delivery device comprising a colloid polymer thin film with nanodispersed nanocarriers and a layer comprising a permeation enhancing agent covering the colloid polymer thin firm, according to another embodiment of the invention.

### DETAILED DESCRIPTION

Embodiments of the invention described herein relate generally to a transmucosal delivery device and a method of manufacturing same, wherein a nanocarrier carrying a biologically active substance is embedded within a colloidal polymer thin film, and a permeation enhancing agent is also embedded in the colloidal polymer thin film or is part of a layer coating the thin film. Such a transmucosal delivery device is intended to deliver the biological active substance to a target location in a two-stage process. First, the colloidal polymer thin film is dissolved when adhered to a mucosal membrane. Once absorbed through the mucosal tissue and into system circulation, the nanocarrier is subject to a number of physiologic effects that will lead to the nanocarrier reaching a target location, such as a target tissue, wherein the biologically active substance dissolves and the biologically active substance payload is released.

The nanocarrier is a nanoparticle (a particle sized sized in at least one dimension to between 1 to 1000 nanometers) which is used as a transport module for the biologically active substance and the mucosal permeation enhancing agent payload complex in some embodiments. The biologically active substance can be a synthetic or a natural material, and for example, can be a cannabis extract comprising cannabinoid and terpene molecules. The mucosal permeation enhancing agent serves to improve delivery of the biologically active substance across buccal mucous membranes, and can be a substance such as bile salts and acids. The nanocarrier 13 with the payload complex is embedded in a colloidal polymer thin film in a dispersed and dehydrated form.

The transmucosal delivery device 10 is manufactured by a process that combines the following precursor materials of the polymeric thin firm 16, biologically active substance, nanocarrier, mucosal permeation enhancing agent, and an optional mucoadhesive enhancing agent:

### Polymeric Thin Film

The colloidal polymer thin film is formed from a polymeric precursor base solution composed of a polymerized water soluble polysaccharide (Pullulan in one embodiment), or combination of polysaccharides (one or more of carboxymethylcellulose, pectin, carrageenan, xanthan gum, locust bean gum), a plasticizer (glycerine in one embodiment), and stabilizers and emulsifiers (one or more of Tween 60, Span 60, propylene glycol), and water.

### Nanocarrier

One suitable nanocarrier is formed from lipid-nanoparticles (LNP), which have a relatively good release profile, targeted drug delivery, and physical stability. LNPs have an average diameter of 40 to 1000 nm and a spherical morphology, and are composed of a lipid component and a surfactant component. The lipid component comprises a solid phase lipid, and can include triglycerides, diglycerides, monoglycerides, fatty acids, steroids, and waxes. The surfactant component comprises a material suitable as an emulsifier and stability enhancer.

Two types of LNPs are solid lipid nanoparticles (SLN) and nanostructured lipid carriers (NLC). Compared to liposomes, SLNs tend to exhibit greater stability and are safer than polymeric carriers because of avoidance of organic solvents during manufacture. SLNs have lipid and surfactant components with compositions that are selected to obtain desired physiochemical properties and qualities, such as particle size and bioactive substance loading.

More particularly, SLNs have a lipid component that is a solid phase at both body and ambient temperature and can be composed of highly purified triglycerides, complex glyceride mixtures, or waxes. In some embodiments, the lipid component can be a solid phase lipid selected from a group consisting of: Tristearin, stearic acid, cetyl palmitate, Precirol®, ATO 5, Compritol®, 888 ATO, Dynasan® 116, Dynasan® 118, Softisan® 154, Cutina® CP, Imwitor® 900 P, Geleol®, Gelot® 64, Emulcire® 61, solid triglycerides, trilaurin, tricaprylin, tripalmitin, tristearin, glyceryl trilaurate, glyceryl trimyristate, glyceryl trimyristin, glyceryl tripalmitate, glyceryl tristearate, glyceryl behenate, glyceryl tribehenin, solid diglycerides, dipalmitin, distearin, solid monoglycerides, glyceryl monostearate, glyceryl palmitostearate, glyceryl stearate citrate, long-chain aliphatic alcohols, cetyl alcohol, stearic alcohol, medium and long-chain fatty acids (C10-C22), stearic acid, palmitic acid, behenic acid, capric acid, fatty alcohol esters with long and medium chain fatty acids with polyols (C10-C22), fatty alcohol esters of long-chain fatty acids, cetyl palmitate, cetearyl olivate, hydroxyoctacosanyl hydroxystearate, sterols, cholesterol, cholesterol esters, cholesteryl hemisuccinate, cholesteryl butyrate, cholesterol palmitate, fatty amines, stearyl amine, waxes, beeswax, shea butter, cocoa butter, carnauba wax, ozokerite wax, paraffin wax, ceramides, hydrogenated vegetable oils, hydrogenated castor oil, quaternary ammonium derivatives, behenyl trimethyl ammonium chloride and/or mixtures thereof.

SLNs have a surfactant component composed of a hydrophilic, lipophilic or amphiphilic material with a concentration between about 0.5 to 5.0% by weight. In some embodiments, the surfactant component is a hydrophilic emulsifier selected from a group consisting of Iuronic® F68 (poloxamer 188) , Pluronic® F127 (poloxamer 407), Tween 20, Tween 40, Tween 80, polyvinyl alcohol, Solutol® HS15, trehalose, sodium deoxycholate, sodium glycocholate, sodium oleate, and polyglycerol methyl glucose distearate. In some other embodiments, the surfactant component is a lipophilic emulsifier selected from a group consisting of Myverol® 18-04K, Span 20, Span 40, and Span 60. In some other embodiments, the surfactant component is an amphiphilic emulsifier selected from a group consisting of egg lecithin, soya lecithin, phosphatidylcholines, phosphatidylethanolamines, and Gelucire® 50/13.

NLCs are differentiated from SLNs by the composition of the solid matrix; in particular, the lipid component in NLCs contain both solid phase and liquid phase lipids at body and ambient temperatures. The liquid phase lipids are selected from a group consisting of: medium chain triglycerides, paraffin oil, 2-octyl dodecenaol, oleic acid, squalene, isopropyl myristate, oils formed by extraction of the oil fraction of plants as well as fish oils, algae oils, marine oils, oils derived from petroleum, short-chain fatty alcohols, medium-chain aliphatic branched fatty alcohols, fatty acid esters with short-chain alcohols, isopropyl myristate, isopropyl palmitate, isopropyl stearate, dibutyl adipate, medium chain triglycerides, capric and caprylic acid triglycerides, Ci2-C16 octanoates, fatty alcohol ethers, Vitamin E , Miglyol® 912, Transcutol® HP, Labrafil Lipofile® WL 1340, Labrafac® PG, Lauroglycol® FCC, and Capryol® 90.

### Biologically Active Substance

In one embodiment, the biologically active substance is a cannabis extract, i.e. a natural chemical product extracted from plants of the genus Cannibis, or a synthetic agonist of cannabinoid receptors. The cannabis extract can be a raw extract of cannabinoids and terpenes from Sativa species, in a concentrated form and typically a viscous oil at room temperature and pressure. This oil typically contains cannabinoids, plant waxes, plant lipids, and plant pigments such as chlorophylls.

The cannabis extract may be further processed to fractionate its constituents, and thereby control the composition of the final distillate. The extraction process may utilize solvents such as ethanol or benzene or other volatiles, including supercritical CO2, or may use solventless methods. The extract may contain tetrahydrocannabinol (THC) in concentrations of 0% to 95%. Cannabinoids such as Cannabidiol (CBD) may be present in amounts between 0 and 95%. Terpenes may be present in amounts between 0% and 25%.

The cannabis constituents may be fractionated into components that then allow an admixture to be formulated that includes a known percentage of each, allowing a customized composition that will be nanoencapsulated and delivered by the buccal thin film.

In other embodiments, the biologically active substance comprises one or more bioactives from a plant, animal, or microorganism extract selected from a group consisting of: iris extract, ashitaba extract, thujopsis dolobrata extract, asparagus extract, avocado extract, sweet hydrangea leaf extract, almond extract, altea extract, arnica extract, aloe extract, apricot extract, apricot kernel extract, ginkgo extract, inchikow extract, fennel extract, turmeric extract, oolong tea extract, uva-ursi extract, rose fruit extract, echinacea leaf extract, isodon japonicus extract, scutellaria root extract, phellodendron bark extract, coptis japonica extract, barley extract, panax ginseng extract, hypericum extract, lamium album extract, ononis extract, Netherland mustard extract, orange extract, a dried seawater product, seaweed extract, persimmon leaf extract, pyracantha fortuneana fruit extract, hydrolyzed elastin, a hydrolyzed wheat powder, hydrolyzed silk, pueraria root extract, chamomilla extract, oil-soluble chamomilla extract, carrot extract, capillary artemisia extract, wild oat extract hibiscus sabdariffa extract, licorice extract, oil-soluble licorice extract, kiwi extract, kiou apple extract, Jew's ear mushroom extract, cinchona bark extract, cucumber extract, paulownia tomentosa leaf extract, guanosine, guava extract, sophora root extract gardenia extract oil, kuma bamboo grass extract, sophora flavescens extract, walnut extract oil, chestnut extract, grapefruit extract, clematis extract, black rice extract, brown sugar extract, black vinegar extract, chlorella extract, mulberry extract, gentian extract, geranium thunbergii extract, black tea extract, yeast extract, magnolia bark extract, coffee extract burdock extract, rice extract, fermented rice extract, fermented rice bran extract, rice germ, comfrey extract, collagen, cowb extract, saisin extract, bupleurum root extract, umbilical cord extract liquid, saffron extract, salvia extract, saponaria officinalis extract, sasa extract, hawthorn extract sansha extract, Japanese pepper extract, shiitake mushroom extract, rehmannia root extract, lithospermum root extract, perilla extract, Japanese linden extract, meadowsweet extract, jatoba extract, peony extract, ginger extract, calamus root extract, white birch extract, white Jew's ear mushroom extract, horsetail extract, stevia extract, a stevia-fermented product, Chinese tamarisk extract, English ivy extract, whitethorn extract, Sambucus nigra extract, yarrow extract, peppermint extract, sage extract, mallow extract, cnidium rhizome extract, Japanese green gentian extract, mulberry bark extract, rhubarb extract, soybean extract, jujube extract, thyme extract, dandelion extract, lichens extract, tea extract, clove extract, imperata extract, citrus unshiu peel extract, tea tree extract, Chinese blackberry extract, capsicum extract, Japanese angelica root extract, calendula officinalis extract, peach kernel extract, spruce extract, houttuynia extract, tomato extract, natto extract, carrot extract, garlic extract, eglantine extract, hibiscus extract, ophiopogon extract, lotus extract, parsley extract, birch extract, honey, hamamelis extract, parietaria extract, isodon japonicus extract, bisabolol, cypress extract, lactobacillus bifidus extract, loquat extract, coltsfoot extract, petasites japonicus extract, hoelen extract, butcher bloom extract, grape extract, grape seed extract, propolis, loofah extract, safflower extract, peppermint extract, linden extract, tree peony extract, hop extract, rosa rugosa extract, pine extract, horse chestnut extract, skunk cabbage extract, soapberry extract, melissa extract, mozuku extract, peach extract, cornflower extract, eucalyptus extract, saxifrage extract, yuzu extract, lily extract, coix seed extract, wormwood extract, lavender extract, green tea extract, egg shell membrane extract, apple extract, rooibos tea extract, litchi extract, lettuce extract, lemon extract, weeping forsythia extract, astragalus sinicus extract, rose extract, rosemary extract, roman chamomile extract, royal jelly extract, and burnet extract.

In other embodiments, the biologically active substance 13 comprises one or more bioactives from a plant-derived polyphenols selected from a group consisting of: acacetin, apiin, apigenin, apigetrin, artoindonesianin P, baicalein, baicalin, chrysin, cynaroside, diosmetin, diosmin, eupatilin, flavoxate, 6-hydroxyflavone, genkwanin, hidrosmin, luteolin, nepetin, nepitrin (nepetin 7-glucoside), nobiletin, orientin (isoorientin), oroxindin, oroxylin A, rhoifolin, scutellarein, scutellarin, tangeritin, techtochrysin, tetuin, tricin, veronicastroside, vitexin (isovitexin), and wogonin, a flavonol including 3-hydroxyflavone, azaleatin, fisetin, galangin, gossypetin, kaempferide, kaempferol, isorhamnetin, morin, myricetin, natsudaidain, pachypodol, quercetin, rhamnazin, rhamnetin, and sophorin, a flavanone including butin, eriodictyol, hesperetin, hesperidin, homoeriodictyol, isosakuranetin, naringenin, naringin, pinocembrin, poncirin, sakuranetin, sakuranin, and sterubin, dihydroquercetin), and aromadedrin, alpinumisoflavone, anagyroidisoflavone A and B, calycosin, daidzein, daidzin, derrubone, di-O-methylalpinumisoflavone, formononetin, genistein, genistin, glycitein, ipriflavone, irigenin, iridin, irilone, 4′-methyl-alpinumisoflavone, 5-O-methylgenistein, luteone, ononin, orobol, pratensein, prunetin, pseudobaptigenin, psi-tectorigenin, puerarin, retusin, tectoridin, tectorigenin, and wighteone, 4-arylcoumarins (neoflavones), 4-arylchromanes, dalbergiones and dalbergiquinols, calophyllolide, coutareagenin, dalbergichromene, dalbergin, and nivetin, afzelechin, arthromerin A, arthromerin B, catechin, epicatechin, epigallocatechin, epicatechin gallate, epigallocatechin gallate, epigallocatechin gallate, epiafzelechin, fisetinidol, gallocatechin, gallocatechin gallate, guibourtinidol, meciadanol (3-O-methylcatechin), mesquitol, propyl gallate, robinetinidol, and thearubigin, apiforol and luteoforol, leucocyanidin, leucodelphinidin, leucomalvidin, leucopelargonidin, leucopeonidin, leucorobinetinidin, melacacidin, antirrhinin, apigeninidin, aurantinidin, capensinidin, chrysanthenin, columnidin, commelinin, cyanidin, 6-hydroxycyanidin, cyanidin-3-(di-p coumarylglucoside)-5-glucoside, cyanosalvianin, delphinidin, diosmetinidin, europinidin, fisetinidin, gesneridin, guibourtinidin, hirsutidin, luteolinidin, malvidin, 5-desoxy-malvidin, malvin, myrtillin, oenin, peonidin, 5-desoxy-peonidin, pelargonidin, petunidin, primulin, protocyanin, protodelphin, pulchellidin, pulchellidin 3-glucoside, pulchellidin 3-rhamnoside, robinetinidin, rosinidin, tricetinidin, tulipanin, violdelphin, protocatechuic acid, gallic acid p-caffeic acid, chlorogenic acid, coumaric acid, cyanidin, pelargonidin, peonidin, peonidin malvidin, quercetin, kaempferol, myricetin, apigenin, luteolin, hesperetin, naringenin, eriodictyol, genistein, glycitein, apigenin, luteolin, resveratrol, curcumin, and curcuminoids.

### Permeation Enhancing Agents

The mucosal permeation enhancing agent may be selected from a group consisting of: 23-lauryl ether, aprotinin, azone, benzalkonium chloride, cetylpyridinium chloride, cetyltrimethylammonium bromide, cyclodextrin, dextran sulfate, lauric acid, lauric acid/propylene glycol, lysophosphatidylcholine, menthol, methoxysalicylate, methyloleate, oleic acid, piperine, bile acids and their salts, phosphatidylcholine, polyoxyetheylene, polysorbate 80, sodium EDTA, sodium glycocholate, sodium glycodeoxycholoate, sodium lauryl sufate, sodium salicylate, sodium taurocholate, sodium taurodeoxycholoate, sulfoxoides, and alkyl glycosides.

### Bilosomes

Bilosome are a type of nanocarrier, and in particular, are bile salt stabilized liposomes, which can be used for enhanced transport or proteins through the gut, or transdermally. Bilosomes loaded with antigens, peptides and other biological materials for purposes of immunization. Additionally or alternatively, the bilosomes can form part of a separate layer that adheres to the surface of the polymer thin film.

In this embodiment, the bilosomes are surface modified carriers whereby bile salts are presented at the surface of the particle to enhance penetration of bioactives through the mucosal membranes of the oral cavity. Bile salts may increase penetration of bioactive substances via the paracellular route through the extraction of membrane protein or lipids, membrane fluidization, the creation of reverse micelles in the membrane, and/or creation of aqueous channels. Bile salts can increase transmembrane transport by disruption of the hemidesmosomes or by binding to Ca+2 in the regions of tight junctions. Bile salts may reduce the viscosity and elasticity of the mucus layer adhering to all mucosal surfaces and consequently increase epithelial membrane permeability. Bile salts have inhibitory effects on mucosal membrane peptidases, preserving proteins from digestive enzymes present in saliva and digestive secretions. Bile acids are steroid acids found in the bile of mammals and other vertebrates. Suitable bile acids include primary bile acids that are synthesized by the liver, and secondary bile acids that result from bacterial actions in the colon. Bile acids are conjugated with taurine or glycine in the liver, and the sodium and potassium salts of these conjugated bile acids are called bile salts. Suitable bile acids and salts include: taurocholic, glycocholic, taurochenodeoxycholic, glycochenodeoxycholic, chenodeoxycholic acid, and deoxycholic acid, lithocholic, as well as cholic, chenodeoxycholic and deoxycholic acids. Additional suitable bile salts include the conjugated salts of their 7-alpha-dehydroxylated derivatives, deoxycholic acid and lithocholic acid, and derivatives of cholic, chenodeoxycholic and deoxycholic acids.

In some embodiments, the nanocarrier may be a nano-scale bilosome (herein referred to as “nano-bilosome”). For example, a nano-bilosome nanocarrier can be based on a sodium deoxycholate (API) formulation with and without benzyl alcohol. As used herein, “bilosomes” are vesicles that comprise non-ionic surfactants and transport enhancing molecules which facilitate the transport of lipid-like molecules across mucosal membranes. In these embodiments, the permeation enhancing agent comprises a bile salt and nano-bilosomes. The bilosomes encapsulate the biologically active substance within a stable structure of 50-500 nanometers in size. The pH ranges from about 8.1 to about 8.5. In one embodiment, the pH of the composition is about 8.1, or alternatively, about 8.2, or alternatively, about 8.3, or alternatively, about 8.4, or alternatively, about 8.5. In another embodiment, the pH of the aqueous solution is about 8.3.

### Mucoadhesive Enhancing Agents

The mucosal thin film delivery device can optionally include agents which enhance the mucoadhesive property of the polymeric thin film. Suitable agents include: Carbopol 971, Carbopol 974, Carbopol 980, Carbopol 940, Carbopol 941, Carbopol 1382, carboxymethlycellulose and hydroxyproplylmethylcelluslose and salts thereof, xanthan gums, polycarbophil, and mixtures thereof. Each would be present in the mixture at 0.01 to 1% of total weight.

### Manufacture

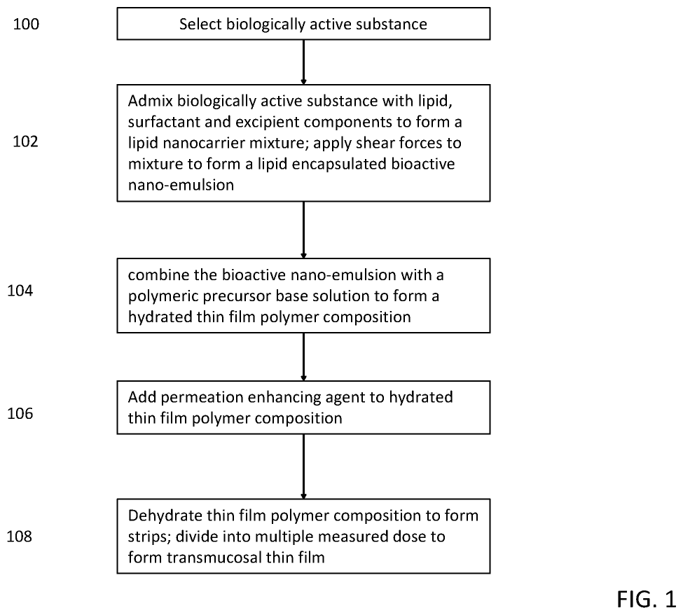

Referring now to FIG. 1, the transmucosal delivery device is manufactured according to the following process:

Select Biologically Active Material. Select one or more of the aforementioned biologically active substances for transmucosal delivery based on a desired physiological effect or therapeutic indication (step 100).

Form Lipid Encapsulated Bioactive Nano-emulsion Incorporate the selected biologically active substance into a lipid nanocarrier by admixing a lipid component, a surfactant component, the biologically active substance and an excipient component to form a lipid nanocarrier mixture. Subject the mixture to shear forces sufficient to create a lipid encapsulated bioactive nano-emulsion in the range of 40-800 nm (step 102). The shear forces can be created by a process selected from: high pressure homogenization in an industrial homogenizer at elevated or low temperatures (hot homogenization at temperatures>100° C. or cold homogenization at temperatures<60° C.), solvent emulsification, evaporation or diffusion, supercritical fluid extraction (of emulsions), and ultrasonication,

Form Hydrated Thin Film Composition. Combine the bioactive nano-emulsion with the polymeric precursor base solution to form a thin film polymer composition in a liquid (hydrated) form (step 104). This can be accomplished using a blade based mixing apparatus or an industrial homogenizer at low speed to avoid air bubble formation.

Add Permeation Enhancing Agent. In a first embodiment, the permeation enhancing agent is added as the final component to the hydrated thin film polymer composition, as a dispersion, in quantities of 0.1 to 2% (w/v) of the final hydrated mixture (step 106). In an alternative second embodiment, the permeation enhancing agent is added to the surface of the thin film during the drying process, as a fine powder which adheres to the strip surface as a layer, rather than being dispersed throughout the dried thin film structure, i.e. embedded in the polymeric thin firm.

Constructing Oral Strips. The thin film polymer composition is then dehydrated to form strips (step 108). The strips are typically between 0.5-2.0 cm square, but other sizes are acceptable. They typically weigh between 60 and 250 mg. This is accomplished by extrusion through valves to create a continuous strip on release paper for cutting into individual dose strips by an automated cutting machine, or on a silicone sheet for hand cutting into individual dose strips.

### Product

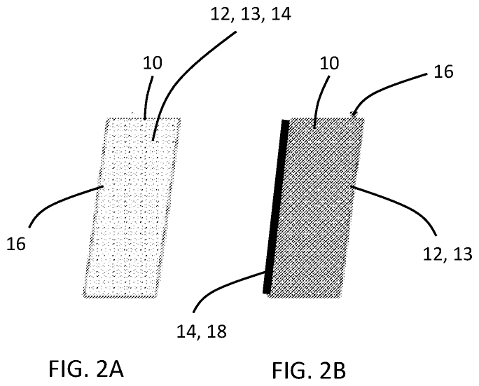

Referring now to FIGS. 2(a) and (b) the transmucosal delivery device 10 generally comprises a mucosal permeation enhancing agent 14 and a nanocarrier 12 containing a biologically active substance 13 in a nanoscale payload complex. The nanocarrier 12 with the payload complex is embedded in a colloidal polymer thin film 16 in a dispersed and dehydrated form. In the first embodiment shown in FIG. 2(a), the permeation enhancing agent is part of the nanocarrier payload complex and is dispersed throughout the polymer thin film 16 with the biologically active substance 13. In the second embodiment shown in FIG. 2(b), the permeation enhancing agent is part of a separate layer 18 that adheres to the surface of the polymer thin film 16.

In some embodiments, the transmucosal delivery device can be provided in the form of thin strips each having an area between 0.5 sq. cm. and 1 sq. cm, a thickness of 0.25-2.5 mm., and a weight between 50 mg and 500 mg. The thin strips can be packaged in a unit dose package (not shown) for end user consumption.

### Use

A strip of the transmucosal delivery device 10 is removed from its unit dose package. The strip is then placed within the space between the gingival buccal mucosa and the cheek buccal mucosa. The device 10 is expected to dissolve with 5 minutes, releasing first permeation enhancing agents and then the biologically active payload, resulting in rapid absorption of nanocarriers containing therapeutic bioactive substances.

### EXAMPLES

The following are non-limiting examples of the described embodiments:

### Example 1

### Nanocarrier Creation Process

1. Measure precursor materials:

| Hemp oil concentrate (bioactive) | 2.7% (27 mg/ml) | (oil phase) |
|---|---|---|
| Olive oil (lipid component) | 1.8% | (oil phase) |
| medium chain triglyceride (MCT) | 1.8% | (oil phase) |
| oil (lipid) | | |
| Tween 60 ™ (surfactant) | 4.2% | (aqueous phase) |
| Span 60 (surfactant) | 2.1% | (oil phase) |
| Water | 87.4% | (aqueous phase) |

2. Combine aqueous phase materials and stir for about 5 minutes at 500 rpm.

3. Combine oil phase materials and stir for about 5 minutes at 500 rpm.

4. Combine oil and aqueous phase materials, stir for about 5 minutes at 500 rpm.

5. Add combined phase materials to room temperature water bath and sonicate at 100% amplitude (90 microns) until solution reaches 40° Celsius (C).

6. Once solution reaches 40° C., remove and add to ice bath.

7. Continue sonicating until solution reaches 60° C., forming lipid encapsulated bioactive nano-emulsion.

### Strip Production Process

1. Measure solid materials:
a. 18% pullulan
b. 0.2% K-carrageenan
c. 0.1% Pectin
d. 0.1% Xanthan gum
e. 0.1% Locust bean gum

2. Measure aqueous materials:
a. 3% glycerin
b. 1% propylene glycol
c. lipid encapsulated bioactive nano-emulsion
d. Additional water to make up 100% of total sample volume

3. Combine solid materials.

4. Combine aqueous materials.

5. Add solid materials to mixer.

6. Mix at slow speed while adding aqueous materials.

7. Mix until homogenous mixture obtained.

8. Place mixture onto drying sheet and spread evenly.

9. Dry strip mixture at appropriate temperature.

10. Apply dry powdered sodium deoxycholate to the surface of the strip.

### Example 2

In this example, the transmucosal delivery device comprises a nano-bilosome nanocarrier, based on a sodium deoxycholate (API) formulation with and without benzyl alcohol.

Such derivatives include, but are not limited to, the “bile acids” cholic acid and chenodeoxycholic acid, their conjugation products with glycine or taurine such as glycocholic and taurocholic acid, derivatives including deoxycholic and ursodeoxycholic acid, and salts of each of these acids.

### Nanobilosome Creation Step

A composition of sodium deoxycholate (0.5% and 1%) is prepared comprising sodium phosphate (10 mM), sodium chloride (75-90 mM), benzyl alcohol (0.9%), deoxycholic acid, pH 8.3

1. Measure precursor materials:

| | | | |
|---|---|---|---|
| a. | Sodium deoxycholate | 2% | (oil phase) |
| b. | Cholesterol | 2% | (oil phase) |
| c. | Tween 60 | 4.2% | (aqueous phase) |
| d. | Span 60 | 2.1% | (oil phase) |
| e. | Water | 87.4% | (aqueous phase) |

2. Combine aqueous phase and stir for about 5 minutes at 500 rpm.

3. Combine oil phase and stir for about 5 minutes at 500 rpm.

4. Combine oil and aqueous phases, stir for about 5 minutes at 500 rpm.

5. Add combined phase to room temperature water bath and sonicate at 100% amplitude (90 microns) until solution reaches 40° Celsius (C).

6. Once solution reaches 40° C., remove and add to ice bath. Continue sonicating until solution reaches 60° C.

Strips are formed by the Strip Production Process from Example 1, minus step 10, wherein the solution produced in step 6 in the Nanobilosome creation step serves as the lipid encapsulated bioactive nano-emulsion.

While particular embodiments have been described in the foregoing, it is to be understood that other embodiments are possible and are intended to be included herein. It will be clear to any person skilled in the art that modifications of and adjustments to the foregoing embodiments, not shown, are possible.

1. A transmucosal delivery device comprising:
a colloidal polymer thin film;
a nanocarrier embedded in the collodial polymer thin firm and carrying a biologically active substance; and
a permeation enhancing agent embedded in or coating the polymer thin film.

2. The transmucosal delivery device as claimed in claim 1, wherein the biologically active material comprises a cannabis extract, or a synthetic agonist of cannabinoid receptors.

3. The transmucosal delivery device as claimed in claim 2, wherein the cannabis extract or synthetic agonist of cannabinoid receptors contain a tetrahydrocannabinol in concentrations of 0% to 95% W/V, a cannabinoid in concentrations between 0 and 95% W/V, and a terpene in concentrations between 0% and 25% W/V.

4. The transmucosal delivery device as claimed in claim 1, wherein the biologically active material comprises a plant, animal, or microorganism extract selected from a group consisting of: iris extract, ashitaba extract, thujopsis dolobrata extract, asparagus extract, avocado extract, sweet hydrangea leaf extract, almond extract, altea extract, arnica extract, aloe extract, apricot extract, apricot kernel extract, ginkgo extract, inchikow extract, fennel extract, turmeric extract, oolong tea extract, uva-ursi extract, rose fruit extract, echinacea leaf extract, isodon japonicus extract, scutellaria root extract, phellodendron bark extract, coptis japonica extract, barley extract, panax ginseng extract, hypericum extract, lamium album extract, ononis extract, Netherland mustard extract, orange extract, a dried seawater product, seaweed extract, persimmon leaf extract, pyracantha fortuneana fruit extract, hydrolyzed elastin, a hydrolyzed wheat powder, hydrolyzed silk, pueraria root extract, chamomilla extract, oil-soluble chamomilla extract, carrot extract, capillary artemisia extract, wild oat extract, hibiscus sabdariffa extract, licorice extract, oil-soluble licorice extract, kiwi extract, kiou apple extract, Jew's ear mushroom extract, cinchona bark extract, cucumber extract, paulownia tomentosa leaf extract, guanosine, guava extract, sophora root extract gardenia extract oil, kuma bamboo grass extract, sophora flavescens extract, walnut extract oil, chestnut extract, grapefruit extract, clematis extract, black rice extract, brown sugar extract, black vinegar extract, chlorella extract, mulberry extract, gentian extract, geranium thunbergii extract, black tea extract, yeast extract, magnolia bark extract, coffee extract burdock extract, rice extract, fermented rice extract, fermented rice bran extract, rice germ, comfrey extract, collagen, cowb extract, saisin extract, bupleurum root extract, umbilical cord extract liquid, saffron extract, salvia extract, saponaria officinalis extract, sasa extract, hawthorn extract sansha extract, Japanese pepper extract, shiitake mushroom extract, rehmannia root extract, lithospermum root extract, perilla extract, Japanese linden extract, meadowsweet extract, jatoba extract, peony extract, ginger extract, calamus root extract, white birch extract, white Jew's ear mushroom extract, horsetail extract, stevia extract, a stevia-fermented product, Chinese tamarisk extract, English ivy extract, whitethorn extract, Sambucus nigra extract, yarrow extract, peppermint extract, sage extract, mallow extract, cnidium rhizome extract, Japanese green gentian extract, mulberry bark extract, rhubarb extract, soybean extract, jujube extract, thyme extract, dandelion extract, lichens extract, tea extract, clove extract, imperata extract, citrus unshiu peel extract, tea tree extract, Chinese blackberry extract, capsicum extract, Japanese angelica root extract, calendula officinalis extract, peach kernel extract, spruce extract, houttuynia extract, tomato extract, natto extract, carrot extract, garlic extract, eglantine extract, hibiscus extract, ophiopogon extract, lotus extract, parsley extract, birch extract, honey, hamamelis extract, parietaria extract, isodon japonicus extract, bisabolol, cypress extract, lactobacillus bifidus extract, loquat extract, coltsfoot extract, petasites japonicus extract, hoelen extract, butcher bloom extract, grape extract, grape seed extract, propolis, loofah extract, safflower extract, peppermint extract, linden extract, tree peony extract, hop extract, rosa rugosa extract, pine extract, horse chestnut extract, skunk cabbage extract, soapberry extract, melissa extract, mozuku extract, peach extract, cornflower extract, eucalyptus extract, saxifrage extract, yuzu extract, lily extract, coix seed extract, wormwood extract, lavender extract, green tea extract, egg shell membrane extract, apple extract, rooibos tea extract, litchi extract, lettuce extract, lemon extract, weeping forsythia extract, astragalus sinicus extract, rose extract, rosemary extract, roman chamomile extract, royal jelly extract, and burnet extract.

5. The transmucosal delivery device as claimed in claim 1, wherein the biologically active material comprises a plant-derived polyphenol selected from a group consisting of: acacetin, apiin, apigenin, apigetrin, artoindonesianin P, baicalein, baicalin, chrysin, cynaroside, diosmetin, diosmin, eupatilin, flavoxate, 6-hydroxyflavone, genkwanin, hidrosmin, luteolin, nepetin, nepitrin (nepetin 7-glucoside), nobiletin, orientin (isoorientin), oroxindin, oroxylin A, rhoifolin, scutellarein, scutellarin, tangeritin, techtochrysin, tetuin, tricin, veronicastroside, vitexin (isovitexin), and wogonin, a flavonol including 3-hydroxyflavone, azaleatin, fisetin, galangin, gossypetin, kaempferide, kaempferol, isorhamnetin, morin, myricetin, natsudaidain, pachypodol, quercetin, rhamnazin, rhamnetin, and sophorin, a flavanone including butin, eriodictyol, hesperetin, hesperidin, homoeriodictyol, isosakuranetin, naringenin, naringin, pinocembrin, poncirin, sakuranetin, sakuranin, and sterubin, dihydroquercetin), and aromadedrin, alpinumisoflavone, anagyroidisoflavone A and B, calycosin, daidzein, daidzin, derrubone, di-O-methylalpinumisoflavone, formononetin, genistein, genistin, glycitein, ipriflavone, irigenin, iridin, irilone, 4′-methyl-alpinumisoflavone, 5-O-methylgenistein, luteone, ononin, orobol, pratensein, prunetin, pseudobaptigenin, psi-tectorigenin, puerarin, retusin, tectoridin, tectorigenin, and wighteone, 4-arylcoumarins (neoflavones), 4-arylchromanes, dalbergiones and dalbergiquinols, calophyllolide, coutareagenin, dalbergichromene, dalbergin, and nivetin, afzelechin, arthromerin A, arthromerin B, catechin, epicatechin, epigallocatechin, epicatechin gallate, epigallocatechin gallate, epigallocatechin gallate, epiafzelechin, fisetinidol, gallocatechin, gallocatechin gallate, guibourtinidol, meciadanol (3-O-methylcatechin), mesquitol, propyl gallate, robinetinidol, thearubigin, apiforol and luteoforol, leucocyanidin, leucodelphinidin, leucomalvidin, leucopelargonidin, leucopeonidin, leucorobinetinidin, melacacidin, antirrhinin, apigeninidin, aurantinidin, capensinidin, chrysanthenin, columnidin, commelinin, cyanidin, 6-hydroxycyanidin, cyanidin-3-(di-p coumarylglucoside)-5-glucoside, cyanosalvianin, delphinidin, diosmetinidin, europinidin, fisetinidin, gesneridin, guibourtinidin, hirsutidin, luteolinidin, malvidin, 5-desoxy-malvidin, malvin, myrtillin, oenin, peonidin, 5-desoxy-peonidin, pelargonidin, petunidin, primulin, protocyanin, protodelphin, pulchellidin, pulchellidin 3-glucoside, pulchellidin 3-rhamnoside, robinetinidin, rosinidin, tricetinidin, tulipanin, violdelphin, protocatechuic acid, gallic acid p- caffeic acid, chlorogenic acid, coumaric acid, cyanidin, pelargonidin, peonidin, peonidin malvidin, quercetin, kaempferol, myricetin, apigenin, luteolin, hesperetin, naringenin, eriodictyol, genistein, glycitein, apigenin, luteolin, resveratrol, curcumin, and curcuminoids.

6. The transmucosal delivery device as claimed in claim 1, consisting of: 23-lauryl ether, aprotinin, azone, benzalkonium chloride, cetylpyridinium chloride, cetyltrimethylammonium bromide, cyclodextrin, dextran sulfate, lauric acid, lauric acid/propylene glycol, lysophosphatidylcholine, menthol, methoxysalicylate, methyloleate, oleic acid, piperine, bile acids and their salts, phosphatidylcholine, polyoxyetheylene, polysorbate 80, sodium EDTA, sodium glycocholate, sodium glycodeoxycholoate, sodium lauryl sufate, sodium salicylate, sodium taurocholate, sodium taurodeoxycholoate, sulfoxoides, and alkyl glycosides.

7. The transmucosal delivery device as claimed in claim 6 wherein the mucosal permeation enhancing agent comprises one or more of bile acids and salts selected from a group consisting of: taurocholic, glycocholic, taurochenodeoxycholic, glycochenodeoxycholic, chenodeoxycholic acid, deoxycholic acid, lithocholic, cholic acid, chenodeoxycholic acid, deoxycholic acid, conjugated salts of their 7-alpha-dehydroxylated derivatives, deoxycholic acid and lithocholic acid, and derivatives of cholic, chenodeoxycholic and deoxycholic acids.

8. The transmucosal delivery device as claimed in claim 1, wherein the nanocarrier comprises lipid-nanoparticles selected from a group consisting of solid lipid nanoparticles and nanostructured lipid carriers.

9. The transmucosal delivery device as claimed in claim 8, wherein the solid lipid nanoparticles comprise a lipid component selected from a group consisting of: Tristearin, stearic acid, cetyl palmitate, Precirol®, ATO 5, Compritol®, 888 ATO, Dynasan® 116, Dynasan® 118, Softisan® 154, Cutina® CP, Imwitor® 900 P, Geleol®, Gelot® 64, Emulcire® 61, solid triglycerides, trilaurin, tricaprylin, tripalmitin, tristearin, glyceryl trilaurate, glyceryl trimyristate, glyceryl trimyristin, glyceryl tripalmitate, glyceryl tristearate, glyceryl behenate, glyceryl tribehenin, solid diglycerides, dipalmitin, distearin, solid monoglycerides, glyceryl monostearate, glyceryl palmitostearate, glyceryl stearate citrate, long-chain aliphatic alcohols, cetyl alcohol, stearic alcohol, medium and long-chain fatty acids (C10-C22), stearic acid, palmitic acid, behenic acid, capric acid, fatty alcohol esters with long and medium chain fatty acids with polyols (C10-C22), fatty alcohol esters of long-chain fatty acids, cetyl palmitate, cetearyl olivate, hydroxyoctacosanyl hydroxystearate, sterols, cholesterol, cholesterol esters, cholesteryl hemisuccinate, cholesteryl butyrate, cholesterol palmitate, fatty amines, stearyl amine, waxes, beeswax, shea butter, cocoa butter, carnauba wax, ozokerite wax, paraffin wax, ceramides, hydrogenated vegetable oils, hydrogenated castor oil, quaternary ammonium derivatives, behenyl trimethyl ammonium chloride, and/or mixtures thereof.

10. The transmucosal delivery device as claimed in claim 8, wherein the solid lipid nanoparticles comprise a surfactant component comprising a hydrophilic emulsifier selected from a group consisting of: luronic® F68 (poloxamer 188) , Pluronic® F127 (poloxamer 407), Tween 20™, Tween 40™, Tween 80™, polyvinyl alcohol, Solutol® HS15, trehalose, sodium deoxycholate, sodium glycocholate, sodium oleate, and polyglycerol methyl glucose distearate.

11. The transmucosal delivery device as claimed in claim 8, wherein the solid lipid nanoparticles comprise a surfactant component comprising a liophilic emulsifier selected from a group consisting of: Myverol® 18-04K, Span 20™, Span 40™, and Span 60™.

12. The transmucosal delivery device as claimed in claim 8, wherein the solid lipid nanoparticles comprise a surfactant component comprising an amphiphilic emulsifier selected from a group consisting of: egg lecithin, soya lecithin, phosphatidylcholines, phosphatidylethanolamines, and Gelucire® 50/13.

13. The transmucosal delivery device as claimed in claim 8, wherein the nanostructured lipid carriers comprise a liquid phase lipid selected from a group consisting of: medium chain triglycerides, paraffin oil, 2-octyl dodecenaol, oleic acid, squalene, isopropyl myristate, oils formed by extraction of the oil fraction of plants and fish oils, algae oils, marine oils, oils derived from petroleum, short-chain fatty alcohols, medium-chain aliphatic branched fatty alcohols, fatty acid esters with short-chain alcohols, isopropyl myristate, isopropyl palmitate, isopropyl stearate, dibutyl adipate, medium chain triglycerides, capric and caprylic acid triglycerides, Ci2-C16 octanoates, fatty alcohol ethers, Vitamin E , Miglyol® 912, Transcutol® HP, Labrafil Lipofile® WL 1340, Labrafac® PG, Lauroglycol® FCC, and Capryol® 90.

14. The transmucosal delivery device as claimed in claim 1, wherein the nanorcarrier is a nano-bilosome.

15. The transmucosal delivery device as claimed in claim 1 further comprising a mucoadhesive enhancing agent embedded in the polymer thin film, and selected from a group consisting of: Carbopol 971, Carbopol 974, Carbopol 980, Carbopol 940, Carbopol 941, Carbopol 1382, carboxymethlycellulose and salts thereof, hydroxyproplylmethylcelluslose and salts thereof, xanthan gums, polycarbophil, and mixtures thereof.

16. A method for manufacturing a transmucosal delivery device comprising:
(a) selecting a biologically active material;
(b) admixing a lipid component, a surfactant component, and the biologically active material to form a lipid nanocarrier mixture;
(c) subjecting the lipid nanocarrier mixture to shear forces sufficient to create a lipid encapsulated bioactive nano-emulsion;
(d) combining the bioactive nano-emulsion with a polymeric precursor base solution to form a hydrated thin film polymer composition; and
(e) dehydrating the thin film polymer composition to form a strip.

17. The method as claimed in claim 16 further comprising between steps (d) and (e): adding a permeation enhancing agent to the hydrated thin film polymer composition.

18. The method as claimed in claim 16 further comprising during step (e):
applying a layer comprising a permeation enhancing agent to the surface of the thin film polymer composition during the dehydrating.

19. The method as claimed in claim 16 wherein in step (b), an excipient component is admixed with the lipid component, surfactant component, and biologically active material.

20. The method as claimed in claim 16 wherein the polymeric precursor base solution is composed of a polymerized water soluble polysaccharide, or combination of polysaccharides, a plasticizer, stabilizers and emulsifiers, and water.

21. The method as claimed in claim 16, wherein the shear forces are provided by a process selected from a group consisting of: high pressure homogenization, solvent emulsification, evaporation or diffusion, supercritical fluid extraction of emulsions, and ultrasonication.
